(19) United States
(12) Patent Application Publication
(10) Pub. No.: US 2025/0120605 A1
(43) Pub. Date: Apr. 17, 2025

(54) SPHYGMOMANOMETER AND METHOD FOR CONTROLLING SPHYGMOMANOMETER

(71) Applicant: OMRON HEALTHCARE Co., Ltd., Kyoto (JP)

(72) Inventor: Yukiya SAWANOI, Kyoto (JP)

(21) Appl. No.: 18/990,584

(22) Filed: Dec. 20, 2024

Related U.S. Application Data
(63) Continuation of application No. PCT/JP2023/018716, filed on May 19, 2023.

(30) Foreign Application Priority Data
Sep. 7, 2022 (JP) 2022-142433

(51) Int. Cl. A61B 5/025; A61B 5/021; A61B 5/022

(52) U.S. Cl. CPC A61B 5/025; A61B 5/02108; A61B 5/02233

(57) ABSTRACT

A sphygmomanometer includes a blood pressure measurement unit that measures a blood pressure of a user based on a pulse wave signal in a pressurization process of increasing a cuff pressure indicating a pressure inside a cuff worn around a measurement site of the user, and a pulse rate measurement unit that measures a pulse rate of the user based on the pulse wave signal in the pressurization process. The blood pressure measurement unit determines whether to continue the pressurization process based on a pulse rate. The sphygmomanometer further includes a monitoring unit that monitors an arrhythmia of the user based on a pulse wave signal in the pressurization process.

### CROSS-REFERENCE TO RELATED APPLICATIONS

This application is the U.S. national stage application filed pursuant to 35 U.S.C. 365(c) and 120 as a continuation of International Patent Application No. PCT/JP2023/018716, filed May 19, 2023, which application claims priority to Japanese Patent Application No. 2022-142433, filed Sep. 7, 2022, which applications are incorporated herein by reference in their entireties.

### TECHNICAL FIELD

The present disclosure relates to a sphygmomanometer and a method for controlling a sphygmomanometer.

### BACKGROUND ART

In the related art, there is known a technique of increasing the accuracy in determination of atrial fibrillation by repeating cuff inflation when atrial fibrillation that is a type of arrhythmia has been detected in blood pressure measurement and repeating the process of determining the presence of atrial fibrillation as described in Patent Document 1 (Chinese Patent No. 107205670).

### CITATION LIST

### Patent Literature

Patent Document 1: Chinese Patent No. 107205670

### SUMMARY OF INVENTION

According to Patent Document 1, it is necessary to repeat the process of determining atrial fibrillation in order to increase the accuracy in determination. For this reason, the time required for measurement becomes longer, and the user feels repeated compression by the cuff that gives a feeling of restraint, which may cause the user to feel troubled.

The present disclosure aims in some respects to provide a sphygmomanometer and a method for controlling a sphygmomanometer that enable to easily and accurately determine whether an arrhythmia has occurred during blood pressure measurement.

In an example of the present disclosure, a sphygmomanometer includes a blood pressure measurement unit that measures a blood pressure of a user based on a pulse wave signal in a pressurization process of increasing a cuff pressure indicating a pressure inside a cuff worn around a measurement site of the user, and a pulse rate measurement unit that measures a pulse rate of the user based on the pulse wave signal in the pressurization process. The blood pressure measurement unit determines whether to continue the pressurization process based on a pulse rate. The sphygmomanometer further includes a monitoring unit that monitors an arrhythmia of the user based on a pulse wave signal in the pressurization process.

According to the above configuration, it is possible to easily and accurately determine whether an arrhythmia has occurred during blood pressure measurement in a pressurization measurement method.

In another example of the present disclosure, the blood pressure measurement unit measures a blood pressure based on a pulse wave signal in the pressurization process if a pulse rate is equal to or higher than a threshold, stops the pressurization process after the measurement, and continues the pressurization process if the pulse rate is lower than the threshold.

According to the above configuration, in the pressurization measurement method, it is possible to obtain pulse rates sufficient for accurately determining whether an arrhythmia has occurred.

In another example of the present disclosure, the blood pressure measurement unit measures a systolic blood pressure based on a pulse wave signal in a pressurization process, stops the pressurization process if the pulse rate is equal to or higher than a threshold at a first timing at which the cuff pressure is increased to a predetermined pressure equal to or higher than a systolic blood pressure, and continues the pressurization process if the pulse rate is lower than the threshold at the first timing.

According to the above configuration, in the pressurization measurement method, it is possible to obtain pulse rates sufficient for accurately determining whether an arrhythmia has occurred.

In another example of the present disclosure, the blood pressure measurement unit stops the pressurization process at a second timing if the pulse rate is equal to or higher than the threshold at the second timing in a period until the cuff pressure reaches a pressure upper limit value higher than the predetermined pressure by continuing the pressurization process, and stops the pressurization process at a timing at which the cuff pressure reaches the pressure upper limit value if the pulse rate is not equal to or higher than the threshold during the period.

According to the above configuration, it is possible to stop the cuff from being excessively pressurized in the pressurization measurement method.

In another example of the present disclosure, a sphygmomanometer includes a blood pressure measurement unit that measures a blood pressure of a user based on a pulse wave signal in a depressurization process of reducing a cuff pressure indicating a pressure inside a cuff worn around a measurement site of the user after a pressurization process of increasing the cuff pressure to a pressure higher than an estimated systolic blood pressure, and a pulse rate measurement unit that measures a pulse rate of the user based on a pulse wave signal in the pressurization process. The blood pressure measurement unit determines whether to continue the pressurization process based on a pulse rate. The sphygmomanometer further includes a monitoring unit that monitors an arrhythmia of the user based on a pulse wave signal in the depressurization process.

According to the above configuration, it is possible to easily and accurately determine whether an arrhythmia has occurred during blood pressure measurement in a depressurization measurement method.

In another example of the present disclosure, the blood pressure measurement unit stops the pressurization process when the cuff pressure reaches a predetermined pressure equal to or higher than the estimated systolic blood pressure if the pulse rate is equal to or higher than a threshold, and continues the pressurization process if the pulse rate is lower than the threshold.

According to the above configuration, in the depressurization measurement method, it is possible to obtain pulse rates sufficient for accurately determining whether an arrhythmia has occurred.

In another example of the present disclosure, the blood pressure measurement unit stops the pressurization process if the pulse rate is equal to or higher than a threshold at a first timing at which the cuff pressure is increased to a predetermined pressure equal to or higher than an estimated systolic blood pressure, and continues the pressurization process if the pulse rate is lower than the threshold at the first timing.

According to the above configuration, in the depressurization measurement method, it is possible to obtain pulse rates sufficient for accurately determining whether an arrhythmia has occurred.

In another example of the present disclosure, the blood pressure measurement unit stops the pressurization process at a second timing if the pulse rate is equal to or higher than the threshold at the second timing in a period until the cuff pressure reaches a pressure upper limit value higher than the predetermined pressure by continuing the pressurization process, and stops the pressurization process at a timing at which the cuff pressure reaches the pressure upper limit value if the pulse rate is not equal to or higher than the threshold during the period.

According to the above configuration, it is possible to stop the cuff from being excessively pressurized in the depressurization measurement method.

In another example of the present disclosure, a method for controlling a sphygmomanometer includes a step of measuring a blood pressure of a user based on a pulse wave signal in a pressurization process of increasing a cuff pressure indicating a pressure inside a cuff worn around a measurement site of the user, a step of measuring a pulse rate of the user based on the pulse wave signal in the pressurization process, a step of determining whether to continue the pressurization process based on the pulse rate, and a step of monitoring an arrhythmia of the user based on the pulse wave signal in the pressurization process.

According to the above configuration, it is possible to easily and accurately determine whether an arrhythmia has occurred during blood pressure measurement in a pressurization measurement method.

In another example of the present disclosure, a method for controlling a sphygmomanometer includes a step of measuring a blood pressure of a user based on a pulse wave signal in a depressurization process of reducing a cuff pressure indicating a pressure inside a cuff worn around a measurement site of the user after a pressurization process of increasing the cuff pressure to a pressure higher than an estimated systolic blood pressure, a step of measuring a pulse rate of the user based on a pulse wave signal in the pressurization process, a step of determining whether to continue the pressurization process based on the pulse rate, and a step of monitoring an arrhythmia of the user based on a pulse wave signal in the depressurization process.

According to the above configuration, it is possible to easily and accurately determine whether an arrhythmia has occurred during blood pressure measurement in a depressurization measurement method.

According to the present disclosure, it is possible to easily and accurately determine whether an arrhythmia has occurred during blood pressure measurement.

### BRIEF DESCRIPTION OF DRAWINGS

FIG. 1 is a diagram illustrating a sphygmomanometer 100 according to the present embodiment.

FIG. 2 is a block diagram illustrating an example of a hardware configuration of the sphygmomanometer.

FIG. 3 is a block diagram illustrating a functional configuration of the sphygmomanometer.

FIG. 4 is a flowchart showing an example of a processing procedure in a pressurization measurement mode of the sphygmomanometer.

FIG. 5 is a flowchart showing another example of the processing procedure in the pressurization measurement mode of the sphygmomanometer.

FIG. 6 is a flowchart showing an example of a processing procedure in a depressurization measurement mode of the sphygmomanometer.

FIG. 7 is a flowchart showing another example of the processing procedure in the depressurization measurement mode of the sphygmomanometer.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

### Application Example

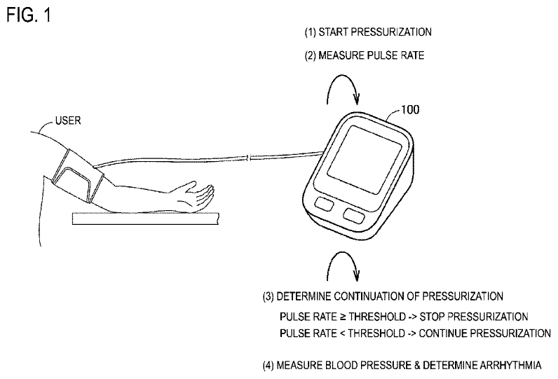

An application example of the present invention will be described with reference to FIG. 1. FIG. 1 is a diagram illustrating a sphygmomanometer 100 according to the present embodiment.

Referring to FIG. 1, the sphygmomanometer 100 is an upper arm-type sphygmomanometer that measures a blood pressure of a subject who is a user. The sphygmomanometer 100 includes a main body and a cuff (arm band) as main components. Note that the sphygmomanometer 100 may be a wrist-type sphygmomanometer in which the main body and the cuff (arm band) are integrated. Hereinafter, the processing contents will be described with reference to FIG. 1.

In FIG. 1, a scene in which a user is using a sphygmomanometer 100 to measure his/her own blood pressure is assumed. The sphygmomanometer 100 is assumed to be a device that measures a blood pressure of a user in a pressurization measurement method in which a blood pressure is measured in a pressurization process of cuff pressure indicating pressure inside a cuff worn around a measurement site (e.g., an arm) of the user.

The sphygmomanometer 100 starts pressurization of the cuff according to a blood pressure measurement instruction of the user (corresponding to (1) of FIG. 1). It is assumed that the pressurization rate of the cuff is constant. The sphygmomanometer 100 measures (counts) the pulse rate based on a detected pulse wave signal in the pressurization process of increasing the cuff pressure (corresponding to (2) in FIG. 1).

Subsequently, the sphygmomanometer 100 determines whether to continue or stop the pressurization process of the cuff pressure based on the pulse rate. Specifically, the sphygmomanometer 100 stops pressurization of the cuff when the measured pulse rate is equal to or higher than a threshold, and continues pressurization of the cuff when the pulse rate is lower than the threshold (corresponding to (3) in FIG. 1).

This is a process for acquiring pulse rates sufficient for accurately detecting an arrhythmia (for example, atrial fibrillation) during blood pressure measurement. Typically, the sphygmomanometer 100 determines whether an arrhythmia has occurred based on an interval (pulse wave interval) between pulse wave signals acquired during blood pressure measurement. Thus, a sufficient number of pulse wave intervals are required in order to ensure the determination being highly accurate. Therefore, the sphygmomanometer 100 executes a process of continuing pressurization of the cuff until the counted pulse rate reaches the threshold or higher.

Then, the sphygmomanometer 100 calculates the blood pressure value of the user and determines whether an arrhythmia has occurred (corresponding to (4) in FIG. 1). In this case, the sphygmomanometer 100 may display the blood pressure value and the arrhythmia determination result of the user on a display.

According to the application example described above, in the process of measuring a blood pressure, pulse rates sufficient for accurately detecting the occurrence of an arrhythmia can be acquired. Therefore, it is possible to accurately determine an arrhythmia while a blood pressure of the user is measured in one blood pressure measurement. In addition, the user does not feel troubled during measurement.

Note that, also when a depressurization measurement method in which a blood pressure is measured in a depressurization process after the pressurization process of the cuff pressure is adopted as a blood pressure measurement method, the cuff is continuously pressurized until the pulse rate reaches a threshold or higher in the pressurization process. For this reason, the cuff pressure at the start of the depressurization process is set to be higher than usual. As a result, assuming that the depressurization rate of the cuff is constant, the pulse rate obtained in the depressurization process becomes higher, and as a result, a sufficient number of pulse wave intervals can be obtained. Therefore, even when the depressurization measurement method is adopted, it is possible to accurately determine an arrhythmia while measuring the blood pressure of the user during one blood pressure measurement.

As described above, according to the sphygmomanometer 100 of the present embodiment, it is possible to easily and accurately determine whether an arrhythmia has occurred during blood pressure measurement.

### Configuration Example

### Hardware Configuration

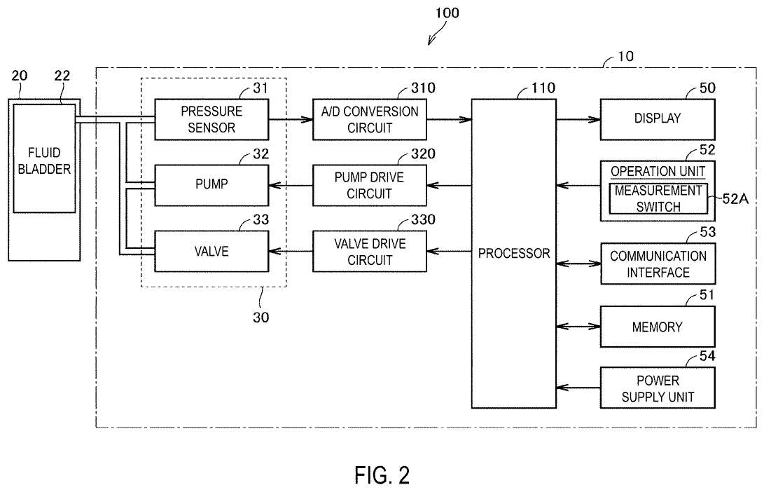

FIG. 2 is a block diagram illustrating an example of a hardware configuration of the sphygmomanometer 100. Referring to FIG. 2, the sphygmomanometer 100 includes a main body 10 and a cuff 20 as main components. A fluid bladder 22 is contained in the cuff 20. The main body 10 includes a processor 110, an air system component 30 for blood pressure measurement, an A/D conversion circuit 310, a pump drive circuit 320, a valve drive circuit 330, a display 50, a memory 51, an operation unit 52, a communication interface 53, and a power supply unit 54.

The processor 110 is an arithmetic processing unit such as a central processing unit (CPU) or a multi processing unit (MPU). By reading and executing a program stored in the memory 51, the processor 110 realizes each of processing operations (steps) of the sphygmomanometer 100 to be described below. For example, the processor 110 performs control to drive a pump 32 and a valve 33 according to an operation signal from the operation unit 52. In addition, the processor 110 calculates a blood pressure value using an algorithm for blood pressure calculation in the oscillometric method and displays the blood pressure value on the display 50.

The memory 51 is realized by a random access memory (RAM), a read-only memory (ROM), a flash memory, or the like. The memory 51 stores a program for controlling the sphygmomanometer 100, data used to control the sphygmomanometer 100, setting data for setting various functions of the sphygmomanometer 100, data of measurement results of blood pressure values, pulse rates, pulse wave intervals, and the like. In addition, the memory 51 is used as a working memory and the like for executing a program.

The air system component 30 supplies or discharges air to or from the fluid bladder 22 included in the cuff 20 through air piping. The air system component 30 includes a pressure sensor 31 for detecting pressure inside the fluid bladder 22, and the pump 32 and the valve 33 as an expansion/contraction mechanism unit for expanding and contracting the fluid bladder 22.

The pressure sensor 31 detects the pressure (cuff pressure) inside the fluid bladder 22 and outputs a signal (cuff pressure signal) corresponding to the detected pressure to the A/D conversion circuit 310. The pressure sensor 31 is, for example, a piezoresistive pressure sensor connected to the pump 32, the valve 33, and the fluid bladder 22 included in the cuff 20 via air piping. The pump 32 supplies air as a fluid to the fluid bladder 22 through the air piping in order to increase the cuff pressure. The valve 33 is opened and closed to control the cuff pressure by discharging air inside the fluid bladder 22 through the air piping or sealing the air in the fluid bladder 22.

The A/D conversion circuit 310 converts an output value of the pressure sensor 31 (for example, a voltage value corresponding to a change in electric resistance caused by a piezoresistive effect) from an analog signal to a digital signal and outputs the digital signal to the processor 110. The processor 110 acquires a signal representing the cuff pressure according to the output value of the A/D conversion circuit 310. The pump drive circuit 320 controls driving of the pump 32 based on a control signal from the processor 110. The valve drive circuit 330 controls opening and closing of the valve 33 based on a control signal from the processor 110.

When a blood pressure is measured in the depressurization measurement method according to a general oscillometric method, the following operation is generally performed. To be specific, the cuff is wrapped around a measurement site (wrist, arm, etc.) of a subject, and during measurement, the pump 32 and the valve 33 are controlled such that the cuff pressure increases above the estimated systolic blood pressure, and then gradually decreases. In the reducing pressure process, the cuff pressure is detected by the pressure sensor, and the variation of arterial volume generated in the artery at the target measurement site is determined to be a pulse wave signal. The maximal blood pressure (systolic blood pressure) and the minimal blood pressure (diastolic blood pressure) are calculated based on the change in amplitude of the pulse wave signal caused by the change in the cuff pressure at that time (mainly, rises and falls).

The display 50 displays various kinds of information including a blood pressure measurement result and the like based on a control signal from the processor 110. The communication interface 53 exchanges various kinds of information with an external device. The power supply unit 54 supplies power to the processor 110 and each piece of hardware.

The operation unit 52 inputs an operation signal corresponding to an instruction from the user to the processor 110. The operation unit 52 includes, for example, a measurement switch 52A for receiving a blood pressure measurement instruction from the user.

### Functional Configuration

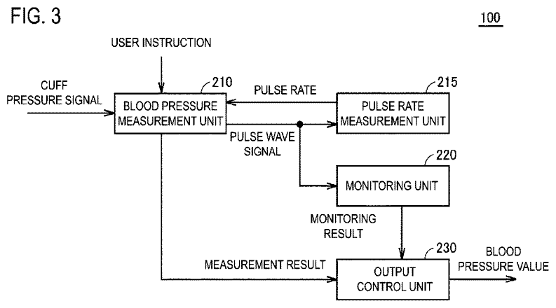

FIG. 3 is a block diagram illustrating a functional configuration of the sphygmomanometer 100. Referring to FIG. 3, the sphygmomanometer 100 includes, as main functional components, a blood pressure measurement unit 210, a pulse rate measurement unit 215, a monitoring unit 220, and an output control unit 230. Each of the functions is realized, for example, by the processor 110 of the sphygmomanometer 1100 executing a program stored in the memory 51. Some or all of these functions may be configured to be realized by hardware.

The blood pressure measurement unit 210 controls the cuff pressure in accordance with a measurement start instruction from the user via the operation unit 52. Specifically, the blood pressure measurement unit 210 performs control to drive the pump 32 via the pump drive circuit 320 and to drive the valve 33 via the valve drive circuit 330. The valve 33 is opened and closed to control the cuff pressure, for example, by discharging air from the fluid bladder 222 or sealing air therein.

The blood pressure measurement unit 210 receives a cuff pressure signal detected by the pressure sensor 31 and extracts a pulse wave signal representing the pulse wave of the measurement site superimposed on the cuff pressure signal. That is, the blood pressure measurement unit 210 detects a pulse wave, which is a pressure component superimposed on the cuff pressure signal in synchronization with the user's heartbeat, from the cuff pressure signal.

The blood pressure measurement unit 210 calculates blood pressure information of the user based on the cuff pressure signal and the pulse wave signal superimposed on the cuff pressure signal. The blood pressure measurement unit 201 measures a blood pressure of a user using, for example, the oscillometric method. Specifically, the blood pressure measurement unit 210 executes, during blood pressure measurement, a pressurization measurement mode in which a blood pressure of the user is measured based on a pulse wave signal in a first pressurization process of increasing the cuff pressure or a depressurization measurement mode in which a blood pressure of the user is measured based on a pulse wave signal in a depressurization process of reducing the cuff pressure after a second pressurization process of increasing the cuff pressure to a pressure higher than an estimated systolic blood pressure.

First, a case in which the blood pressure measurement unit 210 executes the pressurization measurement mode which is a blood pressure measurement mode based on a pressurization measurement method will be described.

When the pressurization measurement mode is executed, the pulse rate measurement unit 215 measures a pulse rate N1 of the user based on a pulse wave signal in the first pressurization process. In this case, the blood pressure measurement unit 210 determines whether to continue the first pressurization process based on the pulse rate N1.

In a certain aspect, the blood pressure measurement unit 210 measures blood pressures (systolic blood pressure and diastolic blood pressure) based on a pulse wave signal in the first pressurization process if the pulse rate N1 is equal to or higher than a threshold Th1, and stops the first pressurization process after the measurement. On the other hand, when the pulse rate N1 is lower than the threshold Th1, the blood pressure measurement unit 210 continues the first pressurization process. Note that, if the cuff pressure reaches a pressure upper limit value Pmax by continuing the first pressurization process, the blood pressure measurement unit 210 stops the first pressurization process at the timing at which the cuff pressure reaches the pressure upper limit value. In this case, the blood pressure measurement unit 210 stops the first pressurization process even if the pulse rate N1 is lower than the threshold Th1. The threshold Th1 is a value determined in advance by a designer or the like of the sphygmomanometer 100.

In another aspect, the blood pressure measurement unit 210 measures blood pressures (systolic blood pressure and diastolic blood pressure) based on a pulse wave signal in the first pressurization process. The blood pressure measurement unit 210 stops the first pressurization process if the pulse rate N1 is equal to or higher than the threshold Th1 at the timing T1 at which the cuff pressure is increased to a pressure P1 equal to or higher than the systolic blood pressure. Typically, the pressure P1 is set to a value higher than the systolic blood pressure by a predetermined value (e.g., 40 mmHg).

On the other hand, if the pulse rate N1 is lower than the threshold Th1 at the timing T1, the blood pressure measurement unit 210 continues the first pressurization process. Subsequently, the blood pressure measurement unit 210 stops the first pressurization process at a timing T2 if the pulse rate N1 is equal to or higher than the threshold Th1 at the timing T2 in the period until the cuff pressure reaches the pressure upper limit value Pmax (P1<Pmax) by continuing the first pressurization process. On the other hand, if the pulse rate N1 is not equal to or higher than the threshold Th1 during the period, the blood pressure measurement unit 210 stops the first pressurization process at the timing at which the cuff pressure reaches the pressure upper limit value Pmax.

The monitoring unit 220 monitors an arrhythmia (for example, atrial fibrillation) of the user based on pulse wave signals in the first pressurization process (that is, determines whether an arrhythmia has occurred). A known method is used as a method for determining whether an arrhythmia has occurred. For example, the monitoring unit 220 determines whether an arrhythmia has occurred based on intervals in which a plurality of pulse waves acquired from the pulse wave signals are generated.

The output control unit 230 displays the measurement result of the blood pressure measurement unit 210 (for example, systolic blood pressure and diastolic blood pressure values) and the monitoring result of the monitoring unit 220 (for example, the determination result of the occurrence of an arrhythmia) on the display 50. Note that the output control unit 230 may transmit the measurement result and the monitoring result to an external device via the communication interface 53, or may output sound via a speaker (not illustrated).

Next, a case in which the blood pressure measurement unit 210 executes the depressurization measurement mode which is a blood pressure measurement mode based on a depressurization measurement method will be described.

When the depressurization measurement mode is executed, the pulse rate measurement unit 215 measures a pulse rate N2 of the user based on a pulse wave signal in the second pressurization process. In this case, the blood pressure measurement unit 210 determines whether to continue the second pressurization process based on the pulse rate N2.

In a certain aspect, if the pulse rate N2 is equal to or higher than a threshold Th2, the blood pressure measurement unit 210 stops the second pressurization process when the cuff pressure reaches a pressure P2 equal to or higher than an estimated systolic blood pressure. Typically, the pressure P2 is set to a value higher than the estimated systolic blood pressure by a predetermined value (e.g., 40 mmHg). On the other hand, if the pulse rate N2 is lower than the threshold Th2, the blood pressure measurement unit 210 continues the second pressurization process. Note that, if the cuff pressure reaches the pressure upper limit value Pmax by continuing the second pressurization process, the blood pressure measurement unit 210 stops the second pressurization process at the timing at which the cuff pressure reaches the pressure upper limit value. The threshold Th2 is a value determined in advance by a designer or the like of the sphygmomanometer.

In another aspect, the blood pressure measurement unit 210 stops the second pressurization process if the pulse rate N2 is equal to or higher than the threshold Th2 at a timing T3 at which the cuff pressure is increased to the pressure P2 equal to or higher than the estimated systolic blood pressure. On the other hand, if the pulse rate N2 is lower than the threshold Th2 at the timing T3, the blood pressure measurement unit 210 continues the second pressurization process. Subsequently, the blood pressure measurement unit 210 stops the second pressurization process at a timing T4 if the pulse rate N2 is equal to or higher than the threshold Th2 at the timing T4 in the period until the cuff pressure reaches the pressure upper limit value Pmax (P3<Pmax) by continuing the second pressurization process. On the other hand, if the pulse rate N2 is not equal to or higher than the threshold Th2 during the period, the blood pressure measurement unit 210 stops the second pressurization process at the timing at which the cuff pressure reaches the pressure upper limit value Pmax.

The monitoring unit 220 monitors an arrhythmia of the user based on a pulse wave signal in the depressurization process. The output control unit 230 displays the measurement result of the blood pressure measurement unit 210 and the monitoring result of the monitoring unit 220 on the display 50.

### Processing Procedure: Pressurization Measurement Mode

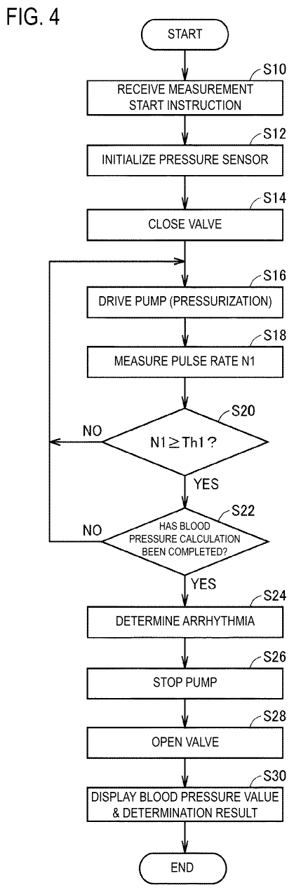

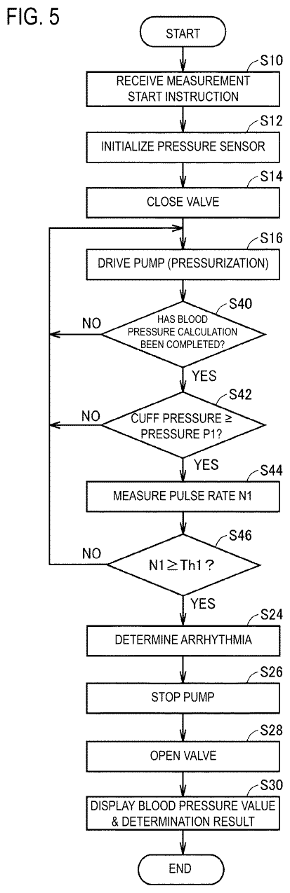

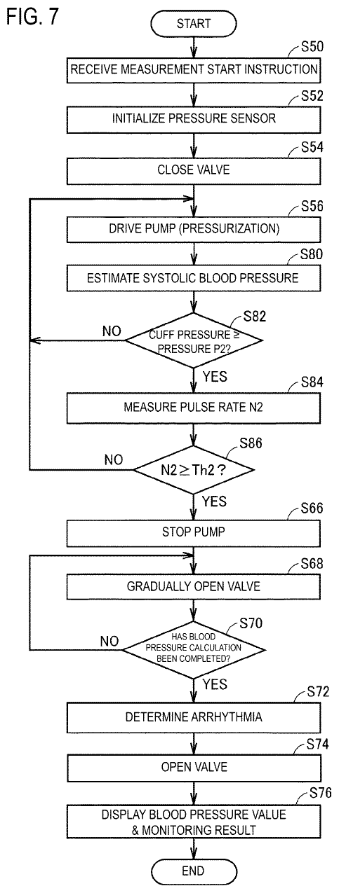

FIG. 4 is a flowchart showing an example of a processing procedure in the pressurization measurement mode of the sphygmomanometer 100. It is assumed that a user is wearing a cuff 20 of the sphygmomanometer 100 at the start of the process. This also applies to FIGS. 5 and 7 to be described below.

Referring to FIG. 4, the processor 110 of the sphygmomanometer 100 receives an instruction to start blood pressure measurement from the user via the measurement switch 52A of the operation unit 52 (step S10). The processor 110 initializes the pressure sensor 31 (step S12). To be more specific, the processor 110 initializes the processing memory area, turns off (stops) the pump 32, and adjusts the pressure sensor 31 to 0 mmHg (sets the atmospheric pressure to 0 mmHg) with the valve 33 being opened.

Next, the processor 110 closes the valve 33 via the valve drive circuit 330 (step S14), and turns on (activates) the pump 32 via the pump drive circuit 320 to start pressurization of the cuff 20 (fluid bladder 22) (step S16). At this time, the processor 110 controls the pressurization rate of the cuff pressure which is the pressure inside the fluid bladder 22 based on the output of the pressure sensor 31 while supplying air from the pump 32 to the fluid bladder 22 through the air piping. Thus, the pressurization process in the pressurization measurement mode is started. Note that, the processor 110 controls the pressurization rate to be constant.

Next, the processor 110 measures (counts) the pulse rate N1 based on a pulse wave signal extracted from a cuff pressure signal detected by the pressure sensor 31 in the pressurization process (step S18). The processor 110 determines whether the pulse rate N1 is equal to or higher than the threshold Th1 (step S20). If the pulse rate N1 is lower than the threshold Th1 (NO in step S20), the processor 110 returns to the processing of step S16 and continues pressurizing the cuff 20 as long as the cuff pressure does not reach the pressure upper limit value Pmax (for example, 300 mmHg). If the pulse rate N1 is equal to or higher than the threshold Th1 (YES in step S20), the processor 110 attempts to calculate the maximal blood pressure (systolic blood pressure) and the minimal blood pressure (diastolic blood pressure), and determines whether the blood pressure calculation has been completed (step S22).

If the blood pressure calculation has not been completed due to insufficient data (NO in step S22), the processor 110 repeats the processing of steps S16 to S22 to continue the pressurization process as long as the cuff pressure does not reach the predetermined pressure upper limit value Pmax.

If the blood pressure calculation has been completed (YES in step S22), the processor 110 determines whether an arrhythmia has occurred based on the pulse wave signal obtained in the pressurization process (step S24). Note that the processing of step S24 may be executed after the processing of step S26 or step S28 to be described below.

Successively, the processor 110 performs control to stop the pump 32 (i.e., stop the pressurization process) (step S26), open the valve 33 (step S28), and discharge the air inside the cuff 20. The processor 110 displays the blood pressure value obtained in step S22 and the determination result obtained in step S24 on the display 50 (step S30).

FIG. 5 is a flowchart showing another example of the processing procedure in the pressurization measurement mode of the sphygmomanometer 100. Referring to FIG. 5, since each processing operation of steps S10 to S16 is the same as described in FIG. 4, the detailed description thereof is not repeated.

After step S16, the processor 110 attempts to calculate the maximal blood pressure (systolic blood pressure) and the minimal blood pressure (diastolic blood pressure) and determines whether the blood pressure calculation has been completed (step S40). If the blood pressure calculation has not been completed (NO in step S40), the processor 110 repeats the processing of steps S16 and S40 to continue the pressurization process as long as the cuff pressure does not reach the pressure upper limit value Pmax.

If the blood pressure calculation has been completed (YES in step S40), the processor 110 determines whether the cuff pressure is equal to or higher than the pressure P1 that is higher than the measured systolic blood pressure (step S42). If the cuff pressure is lower than the pressure P1 (NO in step S42), the processor 110 returns to step S16 and continues the pressurization of the cuff 20. If the cuff pressure is equal to or higher than the pressure P1 (YES in step S42), the processor 110 measures (counts) the pulse rate N1 based on the pulse wave signal in the pressurization process from step S16 (step S44).

The processor 110 determines whether the pulse rate N1 is equal to or higher than the threshold Th1 (step S46). If the pulse rate N1 is lower than the threshold Th1 (NO in step S46), the processor 110 returns to the processing of step S16 and continues the pressurization of the cuff 20 as long as the cuff pressure does not reach the pressure upper limit value Pmax. If the pulse rate N1 is equal to or higher than the threshold Th1 (YES in step S46), the processor 110 performs the processing of step S24. Since each processing operation of steps S24 to S30 is as described in FIG. 4, detailed description thereof is not repeated.

According to the above configuration, since it is possible to obtain pulse rates sufficient for determining whether an arrhythmia has occurred while the blood pressure is measured in the pressurization process in the pressurization measurement mode, the accuracy in the determination can be improved.

### Processing Procedure: Depressurization Measurement Mode

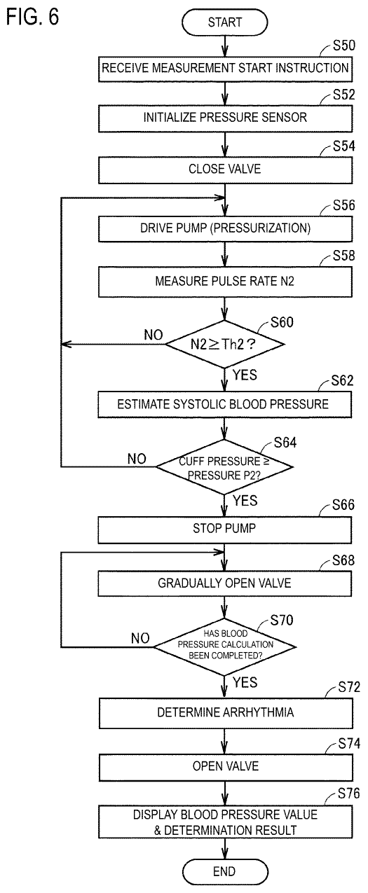

FIG. 6 is a flowchart showing an example of a processing procedure in the depressurization measurement mode of the sphygmomanometer 100.

Referring to FIG. 6, since the processing of steps S50 to S56 is the same as the processing of steps S10 to S16 of FIG. 4, the detailed description thereof is not provided. Note that the pressurization process in the depressurization measurement mode is started by the processing of step S56. At this time, the sphygmomanometer 100 controls the pressurization rate to be constant.

The processor 110 measures the pulse rate N2 based on a pulse wave signal obtained in the pressurization process in the depressurization measurement mode (step S58). The processor 110 determines whether the pulse rate N2 is equal to or higher than the threshold Th2 (step S60). If the pulse rate N2 is lower than the threshold Th2 (NO in step S60), the processor 110 returns to the processing of step S56 and continues the pressurization of the cuff 20 as long as the cuff pressure does not reach the pressure upper limit value Pmax. If the pulse rate N2 is equal to or higher than the threshold Th2 (YES in step S60), the processor 110 estimates the systolic blood pressure based on the pulse wave signal obtained in the pressurization process (step S62). The systolic blood pressure is estimated by using a known method. The processor 110 determines whether the cuff pressure is equal to or higher than the pressure P2 (step S64).

If the cuff pressure is lower than the pressure P2 (NO in step S64), the processor 110 returns to step S56 and continues the pressurization of the cuff 20. If the cuff pressure is equal to or higher than the pressure P2 (YES in step S64), the processor 110 stops the pump 32 (i.e., stops the pressurization process) (step S66) and controls the valve 33 to be gradually opened (step S68). As a result, the cuff pressure gradually decreases from the pressurization process to the depressurization process. At this moment, the processor 110 controls the depressurization rate to be constant.

In the depressurization process, the processor 110 extracts a pulse wave signal from a cuff pressure signal detected by the pressure sensor 31, attempts to calculate the systolic blood pressure and the diastolic blood pressure based on the pulse wave signal, and determines whether the blood pressure calculation has been completed (step S70). If the blood pressure calculation has not been completed (NO in step S70), the processor 110 repeats the processing of steps S68 and S70. If the blood pressure calculation has been completed (YES in step S70), the processor 110 determines whether an arrhythmia has occurred based on the pulse wave signal obtained in the depressurization process (step S72). Note that the processing of step S72 may be executed after the processing of step S74 to be described below.

The processor 110 performs control to fully open the valve 33 (step S74) and rapidly discharge the air inside the cuff 20. The processor 110 displays the blood pressure value obtained in step S70 and the determination result obtained in step S72 on the display 50 (step S76).

FIG. 7 is a flowchart showing another example of the processing procedure in the depressurization measurement mode of the sphygmomanometer 100.

Referring to FIG. 7, since the processing of steps S50 to S56 is the same as the processing of steps S10 to S16 of FIG. 4, the detailed description thereof is not provided. The pressurization process in the depressurization measurement mode is started by the processing of step S56.

The processor 110 estimates the systolic blood pressure based on the pulse wave signal obtained in the pressurization process in the depressurization measurement mode (step S80). The processor 110 determines whether the cuff pressure is equal to or higher than the pressure P2 (step S82).

If the cuff pressure is lower than the pressure P2 (NO in step S82), the processor 110 returns to step S56 and continues the pressurization of the cuff 20. If the cuff pressure is equal to or higher than the pressure P2 (YES in step S82), the processor 110 measures the pulse rate N2 based on the pulse wave signal obtained in the pressurization process (step S84). The processor 110 determines whether the pulse rate N2 is equal to or higher than the threshold Th2 (step S86).

If the pulse rate N2 is lower than the threshold Th2 (NO in step S86), the processor 110 returns to the processing of step S56 and continues the pressurization of the cuff 20 as long as the cuff pressure does not reach the pressure upper limit value Pmax. If the pulse rate N2 is equal to or higher than the threshold Th2 (YES in step S86), the processor 110 performs the processing of step S66. Since each processing operation of steps S66 to S76 is as described in FIG. 6, detailed description thereof is not repeated.

According to the above configuration, since it is possible to obtain pulse rates sufficient for determining whether an arrhythmia has occurred while the blood pressure is measured in the depressurization process in the depressurization measurement mode, the accuracy in the determination can be improved.

### Other Embodiments

(1) In the embodiments described above, a program that causes a computer to function and execute control such as that described in the flowcharts described above may be provided. Such a program can also be provided as a program product stored on a transient computer-readable recording medium attached to a computer, such as a flexible disk, a compact disc read only memory (CD-ROM), a secondary storage device, a main storage device, and a memory card. Alternatively, a program may be provided, which is stored on a recording medium such as a hard disk built into a computer. In addition, the program may also be provided by download via a network.

(2) The configurations given as examples of the embodiments described above are exemplary configurations of the present invention and can be combined with other known technology, and parts thereof may be omitted or modified within the scope not departing from the gist of the present invention. Furthermore, the processes and configurations of other embodiments may be employed as appropriate to the embodiments described above.

### Supplementary Notes

As described above, the present embodiments include the following disclosures.

### Configuration 1

A sphygmomanometer (100) including a blood pressure measurement unit (210) that measures a blood pressure of a user based on a pulse wave signal in a pressurization process of increasing a cuff pressure indicating a pressure inside a cuff worn around a measurement site of the user, and a pulse rate measurement unit (215) that measures a pulse rate of the user based on the pulse wave signal in the pressurization process, in which the blood pressure measurement unit (210) determines whether to continue the pressurization process based on the pulse rate, the sphygmomanometer (100) further including a monitoring unit (220) that monitors an arrhythmia of the user based on the pulse wave signal in the pressurization process.

### Configuration 2

The sphygmomanometer (100) described in configuration 1, in which the blood pressure measurement unit (210) measures a blood pressure based on the pulse wave signal in the pressurization process if the pulse rate is equal to or higher than a threshold, stops the pressurization process after the measurement, and continues the pressurization process if the pulse rate is lower than the threshold.

### Configuration 3

The sphygmomanometer (100) described in configuration 1, in which the blood pressure measurement unit (210) measures a systolic blood pressure based on the pulse wave signal in the pressurization process, stops the pressurization process if the pulse rate is equal to or higher than a threshold at a first timing at which the cuff pressure is increased to a predetermined pressure equal to or higher than the systolic blood pressure, and continues the pressurization process if the pulse rate is lower than the threshold at the first timing.

### Configuration 4

The sphygmomanometer (100) described in configuration 3, in which the blood pressure measurement unit (210) stops the pressurization process at a second timing if the pulse rate is equal to or higher than the threshold at the second timing in a period until the cuff pressure reaches a pressure upper limit value higher than the predetermined pressure by continuing the pressurization process, and stops the pressurization process at a timing at which the cuff pressure reaches the pressure upper limit value if the pulse rate is not equal to or higher than the threshold during the period.

### Configuration 5

A sphygmomanometer (100) including a blood pressure measurement unit (210) that measures a blood pressure of a user based on a pulse wave signal in a depressurization process of reducing a cuff pressure indicating a pressure inside a cuff worn around a measurement site of the user after a pressurization process of increasing the cuff pressure to a pressure higher than an estimated systolic blood pressure, and a pulse rate measurement unit (215) that measures a pulse rate of the user based on the pulse wave signal in the pressurization process, in which the blood pressure measurement unit (210) determines whether to continue the pressurization process based on the pulse rate, the sphygmomanometer (100) further including a monitoring unit (220) that monitors an arrhythmia of the user based on the pulse wave signal in the depressurization process.

### Configuration 6

The sphygmomanometer (100) described in configuration 5, in which the blood pressure measurement unit (210) stops the pressurization process when the cuff pressure reaches a predetermined pressure equal to or higher than the estimated systolic blood pressure if the pulse rate is equal to or higher than a threshold, and continues the pressurization process if the pulse rate is lower than the threshold.

### Configuration 7

The sphygmomanometer (100) described in configuration 5, in which the blood pressure measurement unit (210) stops the pressurization process if the pulse rate is equal to or higher than a threshold at a first timing at which the cuff pressure is increased to a predetermined pressure equal to or higher than the estimated systolic blood pressure, and continues the pressurization process if the pulse rate is lower than the threshold at the first timing.

### Configuration 8

The sphygmomanometer (100) described in configuration 6, in which the blood pressure measurement unit (210) stops the pressurization process at a second timing if the pulse rate is equal to or higher than the threshold at the second timing in a period until the cuff pressure reaches a pressure upper limit value higher than the predetermined pressure by continuing the pressurization process, and stops the pressurization process at a timing at which the cuff pressure reaches the pressure upper limit value if the pulse rate is not equal to or higher than the threshold during the period.

### Configuration 9

A method for controlling a sphygmomanometer (100), including a step of measuring a blood pressure of a user based on a pulse wave signal in a pressurization process of increasing a cuff pressure indicating a pressure inside a cuff worn around a measurement site of the user, a step of measuring a pulse rate of the user based on the pulse wave signal in the pressurization process, a step of determining whether to continue the pressurization process based on the pulse rate, and a step of monitoring an arrhythmia of the user based on the pulse wave signal in the pressurization process.

### Configuration 10

A method for controlling a sphygmomanometer (100), including a step of measuring a blood pressure of a user based on a pulse wave signal in a depressurization process of reducing a cuff pressure indicating a pressure inside a cuff worn around a measurement site of the user after a pressurization process of increasing the cuff pressure to a pressure higher than an estimated systolic blood pressure, a step of measuring a pulse rate of the user based on a pulse wave signal in the pressurization process, a step of determining whether to continue the pressurization process based on the pulse rate, and a step of monitoring an arrhythmia of the user based on a pulse wave signal in the depressurization process.

The embodiments described herein are illustrative in all respects and are not intended as limitations. The scope of the present invention is indicated not by the descriptions above but by the claims and includes all meaning equivalent to the scope and changes within the scope.

### Reference Numerals List

10 Main body
20 Cuff
22 Fluid bladder
30 Air system component
31 Pressure sensor
32 Pump
33 Valve
50 Display
51 Memory
52 Operation unit
52A Measurement switch
53 Communication interface
54 Power supply unit
100 Sphygmomanometer
110 Processor
210 Blood pressure measurement unit
215 Pulse rate measurement unit
220 Monitoring unit
230 Output control unit
310 A/D conversion circuit
320 Pump drive circuit
330 Valve drive circuit.

What is claimed is:

1. A sphygmomanometer comprising:
a blood pressure measurement unit configured to measure a blood pressure of a user based on a pulse wave signal in a pressurization process of increasing a cuff pressure indicating a pressure inside a cuff worn around a measurement site of the user; and
a pulse rate measurement unit configured to measure a pulse rate of the user based on the pulse wave signal in the pressurization process,
wherein the blood pressure measurement unit determines whether to continue the pressurization process based on the pulse rate,
the sphygmomanometer further comprising a monitoring unit configured to monitor an arrhythmia of the user based on the pulse wave signal in the pressurization process.

2. The sphygmomanometer according to claim 1,
wherein the blood pressure measurement unit measures a blood pressure based on the pulse wave signal in the pressurization process if the pulse rate is equal to or higher than a threshold, stops the pressurization process after the measurement, and continues the pressurization process if the pulse rate is lower than the threshold.

3. The sphygmomanometer according to claim 1,
wherein the blood pressure measurement unit measures a systolic blood pressure based on the pulse wave signal in the pressurization process, stops the pressurization process if the pulse rate is equal to or higher than a threshold at a first timing at which the cuff pressure is increased to a predetermined pressure equal to or higher than the systolic blood pressure, and continues the pressurization process if the pulse rate is lower than the threshold at the first timing.

4. The sphygmomanometer according to claim 3,
wherein the blood pressure measurement unit stops the pressurization process at a second timing if the pulse rate is equal to or higher than the threshold at the second timing in a period until the cuff pressure reaches a pressure upper limit value higher than the predetermined pressure by continuing the pressurization process, and stops the pressurization process at a timing at which the cuff pressure reaches the pressure upper limit value if the pulse rate is not equal to or higher than the threshold during the period.

5. A sphygmomanometer comprising:
a blood pressure measurement unit configured to measure a blood pressure of a user based on a pulse wave signal in a depressurization process of reducing a cuff pressure indicating a pressure inside a cuff worn around a measurement site of the user after a pressurization process of increasing the cuff pressure to a pressure higher than an estimated systolic blood pressure; and
a pulse rate measurement unit configured to measure a pulse rate of the user based on the pulse wave signal in the pressurization process,
wherein the blood pressure measurement unit determines whether to continue the pressurization process based on the pulse rate,
the sphygmomanometer further comprising a monitoring unit configured to monitor an arrhythmia of the user based on the pulse wave signal in the depressurization process.

6. The sphygmomanometer according to claim 5,
wherein the blood pressure measurement unit stops the pressurization process when the cuff pressure reaches a predetermined pressure equal to or higher than the estimated systolic blood pressure if the pulse rate is equal to or higher than a threshold, and continues the pressurization process if the pulse rate is lower than the threshold.

7. The sphygmomanometer according to claim 5,
wherein the blood pressure measurement unit stops the pressurization process if the pulse rate is equal to or higher than a threshold at a first timing at which the cuff pressure is increased to a predetermined pressure equal to or higher than the estimated systolic blood pressure, and continues the pressurization process if the pulse rate is lower than the threshold at the first timing.

8. The sphygmomanometer according to claim 7,
wherein the blood pressure measurement unit stops the pressurization process at a second timing if the pulse rate is equal to or higher than the threshold at the second timing in a period until the cuff pressure reaches a pressure upper limit value higher than the predetermined pressure by continuing the pressurization process, and stops the pressurization process at a timing at which the cuff pressure reaches the pressure upper limit value if the pulse rate is not equal to or higher than the threshold during the period.

9. A method for controlling a sphygmomanometer, the method comprising:
measuring a blood pressure of a user based on a pulse wave signal in a pressurization process of increasing a cuff pressure indicating a pressure inside a cuff worn around a measurement site of the user;
measuring a pulse rate of the user based on the pulse wave signal in the pressurization process;
determining whether to continue the pressurization process based on the pulse rate; and
monitoring an arrhythmia of the user based on the pulse wave signal in the pressurization process.

10. A method for controlling a sphygmomanometer, the method comprising:
measuring a blood pressure of a user based on a pulse wave signal in a depressurization process of reducing a cuff pressure indicating a pressure inside a cuff worn around a measurement site of the user after a pressurization process of increasing the cuff pressure to a pressure higher than an estimated systolic blood pressure;
measuring a pulse rate of the user based on the pulse wave signal in the pressurization process;
determining whether to continue the pressurization process based on the pulse rate; and
monitoring an arrhythmia of the user based on the pulse wave signal in the depressurization process.
